(19) United States
(12) Patent Application Publication
(10) Pub. No.: US 2012/0114586 A1
(43) Pub. Date: May 10, 2012

(54) HAIR CONDITIONERS COMPRISING SELECTED CATIONIC SILICONES AND A B SERIES VITAMIN

(75) Inventor: Marcus Krüger, Ellerhoop (DE)

(73) Assignee: Henkel AG & Co. KGaA, Dusseldorf (DE)

(21) Appl. No.: 13/354,497

(22) Filed: Jan. 20, 2012

Related U.S. Application Data
(63) Continuation of application No. PCT/EP2010/059639, filed on Jul. 6, 2010.

(30) Foreign Application Priority Data
Jul. 23, 2009 (DE) 10 2009 027 965.2

(51) Int. Cl. A61K 8/898; A61Q 5/00

(52) U.S. Cl. 424/70.122

(57) ABSTRACT

The invention relates to hair preparations containing selected cationic silicones and at least one B series vitamin as well as to the use of said preparations for the treatment of keratin fibers, in particular human hair.

### CROSS-REFERENCES TO RELATED APPLICATIONS

This application is a continuation of PCT/EP2010/059639, filed on Jul. 6, 2010, which claims priority under 35 U.S.C. §119 to DE 10 2009 027 965.2 filed on Jul. 23, 2009, both of which are hereby incorporated by reference.

### FIELD OF THE INVENTION

The present invention generally relates to hair-treatment agents containing selected cationic silicones and at least one B series vitamin, as well as to the use of these agents for the treatment of keratinic fibers, in particular human hair.

### BACKGROUND OF THE INVENTION

Not least owing to the severe stressing of hair, for example through dyeing or permanent waves as well as through cleaning the hair with shampoos and through environmental pollution, the importance of conditioning products with as long-lasting an action as possible is increasing.

The active substances that are available generally act preferentially on the hair surface. However, the known active substances are not able to cover all requirements to a sufficient extent. A need therefore still exists for active substances or combinations of active substances for cosmetic agents with good conditioning properties and good biodegradability. In formulations containing dyes and/or electrolytes in particular, there is a need for additional active conditioning substances that can be readily incorporated into known formulations. In particular, there is a need for conditioning substances in the agents which significantly improve the combability of wet and dry hair, the handle of wet and dry hair and the gloss.

In hair-conditioning agents, in addition to vitamins, silicones are also used. Among the silicones, in particular amino-functional silicones have established themselves as being suitable. However, the use of the conventional amino-functional silicones leads to a significant loading of keratinic fibers. As a result, subsequent hair styling can become more difficult.

### BRIEF SUMMARY OF THE INVENTION

A combination of active substances comprising an amino-functional silicone and at least one B series vitamin, together with a cosmetic support, achieves most particularly advantageous results in overcoming the previously-noted problems. When this combination is used, surprisingly good properties of the treated keratinic fibers, in particular human hair, are obtained, in particular improved combability, improved gloss and improved elasticity as well as significantly increased wash resistance of dyed hair and longer durability, at the same time as better reshaping performance in waving procedures such as a water wave and permanent wave. A highly preferred combination of active substances also contains, in addition to a cationic, amino-functional silicone and at least one B series vitamin, at least one dimethicone and/or at least one cosmetic oil.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention firstly provides a composition for the treatment of keratinic fibers, containing a) a cationic amino-functional silicone, b) at least one B series vitamin and c) a cosmetic support.

The agents according to the invention contain a combination of active substances comprising at least two components, components a) and b) being used within a specific weight ratio to one another. In preferred agents according to the invention, the weight ratio of imidazoline derivatives a) of formula I to a cationic amino-functional silicone b) is 20:1 to 1:20, more preferably 10:1 to 1:10, particularly preferably 5:1 to 1:5 and in particular 2.5:1 to 1:2.5.

Hair-treatment agents within the meaning of the present invention are, for example, hair colorants, blonding agents, hair shampoos, hair conditioners, conditioning shampoos, hairsprays, hair rinses, deep conditioners, hair masques, hair tonics, permanent wave fixing solutions, hair coloring shampoos, hair fixatives, hair-setting compositions, hairstyling preparations, blow-drying lotions, styling mousses, hair gels, hair waxes or combinations thereof. Preferred agents according to the invention are shampoos, conditioning agents or hair tonics.

Combability is understood according to the invention to mean both the combability of wet fibers and the combability of dry fibers. The combing work or the force exerted during the combing operation of a fiber collective serves as a measure of combability. The measuring parameters can be subject to sensory evaluation by the person skilled in the art or can be quantified by measuring equipment.

Handle is defined as the tactility of a fiber collective, the parameters of fullness and smoothness of the collective being felt and subjected to sensory evaluation by the person skilled in the art.

Shaping is understood to mean the ability to impart a change in shape to a collective of previously treated, keratin-containing fibers, in particular human hair. In hair cosmetics, the term stylability is also used.

Suitable as the cosmetic support according to the invention are, in particular, creams, emulsions, gels or foaming solutions containing surfactants, such as for example shampoos, foam aerosols or other preparations that are particularly suitable for application to the hair. However, it is also conceivable to integrate the ingredients into a formulation in powdered or tablet form, which is dissolved in water before application. The cosmetic supports can in particular be aqueous or aqueous-alcoholic. An aqueous cosmetic support contains at least 50 wt. % water.

Aqueous-alcoholic cosmetic supports are understood within the meaning of the present invention to be aqueous solutions containing 3 to 70 wt. % of a C₁-C₆ alcohol, in particular methanol, ethanol or propanol, isopropanol, butanol, isobutanol, tert.-butanol, n-pentanol, isopentanols, n-hexanol, isohexanols, glycol, glycerol, 1,2-pentanediol, 1,5-pentanediol, 1,2-hexanediol or 1,6-hexanediol. The agents according to the invention can additionally contain other organic solvents, such as for example methoxybutanol, benzyl alcohol, ethyl diglycol or 1,2-propylene glycol. All water-soluble organic solvents are preferred here.

As ingredient a), the agents according to the invention contain a cationic amino-functional silicone polymer. EP 1887024 A1 describes novel cationic amino-functional silicones, which in particular improve the gloss in agents for the care of surfaces, for example human hair. These cationic silicone polymers are distinguished by the fact that they have a silicone backbone and at least one polyether section and also at least one section with an ammonium structure. Examples of the preferred cationic silicone polymers within the meaning of the present invention are also, in addition to the compounds of the above-mentioned EP 1887024 A1, in particular the compounds with the INCI names: Silicone Quaternium-1, Silicone Quaternium-2, Silicone Quatemium-3, Silicone Quaternium-4, Silicone Quaternium-5, Silicone Quatemium-6, Silicone Quaternium-7, Silicone Quatemium-8, Silicone Quaternium-9, Silicone Quatemium-10, Silicone Quatemium-11, Silicone Quatemium-12, Silicone Quatemium-15, Silicone Quatemium-16, Silicone Quaternium-17, Silicone Quatemium-18, Silicone Quaternium-20, Silicone Quaternium-21, Silicone Quaternium-22 and Silicone Quaternium-2 Panthenol Succinate and Silicone Quatemium-16/Glycidyl Dimethicone Crosspolymer. Most preferred is, in particular, Silicone Quaternium-22. This raw material is marketed for example by Evonik with the trade name Abil® T-Quat 60.

The cationic amino-functional silicone polymers are contained in the compositions according to the invention in amounts of 0.01 to 20 wt. %, preferably in amounts of 0.05 to 10 wt. % and most particularly preferably in amounts of 0.1 to 7.5 wt. %. The best results of all are obtained with amounts of 0.1 to 5 wt. %, based in each case on the overall composition of the respective agent.

As ingredient b), the agents according to the invention contain at least one B series vitamin. The vitamin B group or the vitamin B complex includes, inter alia:

Vitamin B₁ (thiamin)
Vitamin B₂ (riboflavin)
Vitamin B₃. This name often includes the compounds nicotinic acid and nicotinamide (niacinamide). Nicotinamide, which is contained in the agents used according to the invention preferably in amounts of 0.05 to 1 wt. %, based on the total agent, is preferred according to the invention.
Vitamin B₅ (pantothenic acid, panthenol and pantolactone). Within the framework of this group, panthenol and/or pantolactone is preferably used. Derivatives of panthenol that can be used according to the invention are, in particular, the esters and ethers of panthenol as well as cationically derivatized panthenols. Individual representatives are, for example, panthenol triacetate, panthenol monoethyl ether and the monoacetate thereof as well as cationic panthenol derivatives. Pantothenic acid is preferably used as a derivative in the form of the more stable calcium salts and sodium salts (Ca pantothenate, Na pantothenate) in the present invention.
Vitamin B₆ (pyridoxine as well as pyridoxamine and pyridoxal).

Panthenol, pantolactone, pantothenic acid, pyridoxine and derivatives thereof as well as nicotinamide are particularly preferred. The above compounds of the vitamin B type, in particular vitamins B₃, B₅ and B₆, are contained in the agents according to the invention preferably in amounts of 0.05-10 wt. %, based on the total agent. Amounts of 0.1-5 wt. % are particularly preferred.

The compositions according to the invention preferably contain further ingredients. These are, for example, particularly preferably ester oils. The ester oils are defined as follows:

Ester oils are understood to be the esters of C₆-C₃₀ fatty acids with C₂-C₃₀ fatty alcohols. Preferred are the monoesters of the fatty acids with alcohols having 2 to 24 C atoms. Examples of fatty acid portions used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Examples of the fatty alcohol portions in the ester oils are isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol as well as technical mixtures thereof. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), hexyl laurate (Cetiol® A), di-n-butyl adipate (Cetiol® B), myristyl myristate (Cetiol® MM), cetearyl isononanoate (Cetiol® SN), decyl oleate (Cetiol® V).

The ester oils can, of course, also be alkoxylated with ethylene oxide, propylene oxide or mixtures of ethylene oxide and propylene oxide. The alkoxylation can be found either on the fatty alcohol part or on the fatty acid part, or on both sections of the ester oils. However, it is preferred according to the invention if the fatty alcohol has been alkoxylated first and then esterified with fatty acid. In the formula (D4-II), these compounds are illustrated in general.

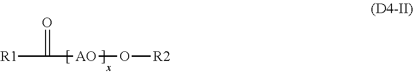

R1 here denotes a saturated or unsaturated, branched or unbranched, cyclic saturated cyclic unsaturated acyl residue with 6 to 30 carbon atoms,

AO denotes ethylene oxide, propylene oxide or butylene oxide,
X denotes a number between 1 and 200, preferably 1 and 100, particularly preferably between 1 and 50, more particularly preferably between 1 and 20, extremely preferably between 1 and 10 and most preferably between 1 and 5,
R2 denotes a saturated or unsaturated, branched or unbranched, cyclic saturated cyclic unsaturated alkyl, alkenyl, alkynyl, phenyl or benzyl residue with 6 to 30 carbon atoms. Examples of fatty acid portions used as residue R1 in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. Examples of the fatty alcohol portions as residue R2 in the ester oils are benzyl alcohol, isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol as well as technical mixtures thereof. A particularly preferred ester oil according to the invention is available, for example, with the INCI name PPG-3 Benzyl Ether Myristate.

The following are also to be understood as ester oils:
dicarboxylic acid esters, such as di-n-butyl adipate, di-(2-ethylhexyl)adipate, di-(2-ethylhexyl)succinate and di-isotridecyl acetate as well as diol esters, such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate, as well as
symmetrical, non-symmetrical or cyclic esters of carbonic acid with fatty alcohols, for example glycerol carbonate or dicaprylyl carbonate (Cetiol® CC),
trifatty acid esters of saturated and/or unsaturated linear and/or branched fatty acids with glycerol,
fatty acid partial glycerides, i.e. monoglycerides, diglycerides and technical mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Oleic acid monoglycerides are preferably used.

The ester oils are used in the agents according to the invention in an amount of 0.01 to 20 wt. %, preferably 0.01 to 10.0 wt. %, particularly preferably 0.01 to 7.5 wt. %, most preferably 0.1 to 5.0 wt. %. It is, of course, also possible according to the invention to use several ester oils at the same time.

In addition, it is also possible to use cosmetic oils with the combination of active substances (A) according to the invention. These oil substances preferably have a melting point of less than 50° C., particularly preferably less than 45° C., more particularly preferably less than 40° C., extremely preferably less than 35° C. and the cosmetic oils are most preferably flowable at a temperature of less than 30° C. These oils are defined and described in more detail below.

The natural and synthetic cosmetic oils include, for example:
vegetable oils. Examples of these oils are sunflower oil, olive oil, soybean oil, rapeseed oil, almond oil, jojoba oil, orange oil, wheat germ oil, peach kernel oil and the liquid portions of coconut oil. However, other triglyceride oils, such as the liquid portions of beef tallow and synthetic triglyceride oils, are also suitable.
liquid paraffin oils, isoparaffin oils and synthetic hydrocarbons as well as di-n-alkyl ethers with a total of between 12 and 36 C atoms, in particular 12 to 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert.-butyl-n-octyl ether, isopentyl-n-octyl ether and 2-methylpentyl-n-octyl ether. The compounds 1,3-di-(2-ethylhexyl)cyclohexane (Cetiol® S) and di-n-octyl ether (Cetiol® OE), which are available as commercial products, may be preferred.

Suitable as natural oils are, for example, amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, maize oil, almond oil, manila oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil.

In many cases, the agents contain at least one surface-active substance, with anionic as well as zwitterionic, ampholytic, nonionic and cationic surface-active substances being suitable in principle. The choice of surface-active substances depends on the nature of the agent. In the case of a shampoo, in particular at least one surfactant from the group of the anionic, zwitterionic or nonionic surface-active substances is selected. It is preferred here that at least one anionic and at least one zwitterionic surface-active substance is selected. Particularly preferably, these surface-active substances are selected from the group of the particularly mild surface-active substances. The ratio between anionic and zwitterionic surface-active substances is preferably between 10:1 and 1:5. The ratio is particularly preferably 5:1 to 1:2.

Suitable as anionic surfactants (Tanion) in preparations according to the invention are all anionic surface-active substances suitable for use on the human body. Typical examples of anionic surfactants are:
linear and branched fatty acids with 8 to 30 C atoms (soaps),
ether carboxylic acids of the formula R—O—(CH₂—CH₂O)ₓ—CH₂—COOH, in which R is a linear alkyl group with 8 to 30 C atoms and x=0 or 1 to 16, and salts thereof,
acyl sarcosides with 8 to 24 C atoms in the acyl group,
acyl taurides with 8 to 24 C atoms in the acyl group,
acyl isethionates with 8 to 24 C atoms in the acyl group,
sulfosuccinic acid mono- and dialkyl esters with 8 to 24 C atoms in the alkyl group and sulfosuccinic acid monoalkyl polyoxyethyl esters with 8 to 24 C atoms in the alkyl group and 1 to 6 oxyethyl groups,
linear alkanesulfonates with 8 to 24 C atoms,
linear alpha-olefin sulfonates with 8 to 24 C atoms,
alpha-sulfo fatty acid methyl esters of fatty acids with 8 to 30 C atoms,
alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12,
hydroxy sulfonates substantially corresponding to at least one of the following two formulae or mixtures thereof as well as salts thereof, CH₃—(CH₂)_{y}—CHOH—(CH₂)ₚ—(CH—SO₃M)-(CH₂)_{z}—CH₂—O—(CₙH₂ₙO)ₓ—H, and/or CH₃—(CH₂)_{y}—(CH—SO₃M)-(CH₂)ₚ—CHOH—(CH₂)_{z}—CH₂—O—(CₙH₂ₙO)ₓ—H, wherein in both formulae y and z=0 or integers from 1 to 18, p=0, 1 or 2 and the sum of (y+z+p) is a number from 12 to 18, x=0 or a number from 1 to 30 and n is an integer from 2 to 4, and M=H or alkali, in particular sodium, potassium, lithium, alkaline earth, in particular magnesium, calcium, zinc and/or an ammonium ion, which may optionally be substituted, in particular mono-, di-, tri- or tetraammonium ions with C₁ to C₄ alkyl, alkenyl or aryl residues,
sulfated hydroxyalkyl polyethylene and/or hydroxyalkylene propylene glycol ethers of the formula R¹—(CHOSO₃M)-CHR³—(OCHR⁴—CH₂)ₙ—OR² with R¹ a linear alkyl residue with 1 to 24 C atoms, R² denotes a linear or branched, saturated alkyl residue with 1 to 24 C atoms, R³ denotes hydrogen or a linear alkyl residue with 1 to 24 C atoms, R⁴ denotes hydrogen or a methyl residue and M denotes hydrogen, ammonium, alkylammonium, alkanol ammonium, wherein the alkyl and alkanol residues each have 1 to 4 C atoms, or a metal atom selected from lithium, sodium, potassium, calcium or magnesium and n denotes a number in the range from 0 to 12 and furthermore the total number of C atoms contained in R¹ and R³ is 2 to 44,
sulfonates of unsaturated fatty acids with 8 to 24 C atoms and 1 to 6 double bonds,
esters of tartaric acid and citric acid with alcohols, which represent addition products of about 2-15 molecules of ethylene oxide and/or propylene oxide to fatty alcohols with 8 to 22 C atoms,
alkyl and/or alkenyl ether phosphates of the formula R¹(OCH₂CH₂)ₙ—O—(PO—OX)—OR², in which R¹ preferably denotes an aliphatic hydrocarbon residue with 8 to 30 carbon atoms, R² denotes hydrogen, a (CH₂CH₂O)ₙR² residue or X, n denotes numbers from 1 to 10 and X denotes hydrogen, an alkali metal or alkaline earth metal or NR³R⁴R⁵R⁶, with R³ to R⁶ independently of one another denoting hydrogen or a C₁ to C₄ hydrocarbon residue,
sulfated fatty acid alkylene glycol esters of the formula RCO(AIkO)ₙSO₃M
in which RCO— denotes a linear or branched, aliphatic, saturated and/or unsaturated acyl residue with 6 to 22 C atoms, Alk denotes CH₂CH₂, CHCH₃CH₂ and/or CH₂CHCH₃, n denotes numbers from 0.5 to 5 and M denotes a metal, such as alkali metal, in particular sodium, potassium, lithium, alkaline earth metal, in particular magnesium, calcium, zinc, or ammonium ion, such as ⁺NR³R⁴R⁵R⁶, with R³ to R⁶ independently of one another denoting hydrogen or a C₁ to C₄ hydrocarbon residue,
monoglyceride sulfates and monoglyceride ether sulfates of the formula: R⁸OC —(OCH₂CH₂)ₓ—OCH₂—[CHO(CH₂CH₂O)_{y}H]—CH₂O(CH₂CH₂O)_{z}—SO₃X,
in which R⁸CO denotes a linear or branched acyl residue with 6 to 22 carbon atoms, x, y and z in total denote 0 or numbers from 1 to 30, preferably 2 to 10, and X denotes an alkali metal or alkaline earth metal. Typical examples of suitable monoglyceride(ether)sulfates within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as the ethylene oxide adducts thereof with sulfur trioxide or chlorosulfonic acid in the form of their sodium salts. Monoglyceride sulfates are preferably used in which R⁸CO denotes a linear acyl residue with 8 to 18 carbon atoms,
amide ether carboxylic acids, R¹—CO—NR²—CH₂CH₂—O—(CH₂CH₂O)ₙCH₂COOM, with R¹ as a straight-chain or branched alkyl or alkenyl residue with a number of carbon atoms in the chain of 2 to 30, n denotes an integer from 1 to 20 and R² denotes hydrogen, a methyl, ethyl, propyl, isopropyl, n-butyl, t-butyl or isobutyl residue and M denotes hydrogen or a metal such as alkali metal, in particular sodium, potassium, lithium, alkaline earth metal, in particular magnesium, calcium, zinc, or an ammonium ion, such as ⁺NR³R⁴R⁵R⁶, with R³ to R⁶ independently of one another denoting hydrogen or a C₁ to C₄ hydrocarbon residue. Products of this type are obtainable, for example, from ChemY with the product name Akypo®.
acyl glutamates of the formula XOOC—CH₂CH₂CH(C(NH)OR)—COOX, in which RCO denotes a linear or branched acyl residue with 6 to 22 carbon atoms and 0 and/or 1, 2 or 3 double bonds and X denotes hydrogen, an alkali metal and/or alkaline earth metal, ammonium, alkylammonium, alkanol ammonium or glucammonium,
condensation products of a water-soluble salt of a water-soluble protein hydrolyzate with a C₈-C₃₀ fatty acid. Products of this type have been commercially available for a long time with the trade name Lamepon®, Maypon®, Gluadin®, Hostapon® KCG or Amisoft®.
alkyl and/or alkenyl oligoglycoside carboxylates, sulfates, phosphates and/or isethionates,
acyl lactylates and
hydroxy mixed ether sulfates.

Where the mild anionic surfactants contain polyglycol ether chains, it is most particularly preferred for these to have a narrow homolog distribution. Furthermore, in the case of mild anionic surfactants with polyglycol ether units, it is preferred for the number of glycol ether groups to be 1 to 20, preferably 2 to 15, particularly preferably 2 to 12. Particularly mild anionic surfactants with polyglycol ether groups without a restricted homolog distribution can also be obtained, for example, if on the one hand the number of polyglycol ether groups is 4 to 12 and Zn or Mg ions are selected as counter-ion. An example of this is the commercial product Texapon® ASV.

Particularly suitable zwitterionic surfactants are the so-called betaines as well as N-alkyl-N,N-dimethylammonium glycinates, for example cocoalkyl dimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines with in each case 8 to 18 C atoms in the alkyl or acyl group, as well as cocoacylaminoethylhydroxyethylcarboxymethyl glycinate. A preferred zwitterionic surfactant is the fatty acid amide derivative known under the INCI name Cocamidopropyl Betaine.

Ampholytic surfactants (Tampho) are understood to be those surface-active compounds that are capable of forming internal salts. Examples of suitable ampholytic surfactants are N-alkyl glycines, N-alkyl propionic acids, N-alkylamino butyric acids, N-alkylimino dipropionic acids, N-hydroxyethyl-N-alkylamidopropyl glycines, N-alkyl taurines, N-alkyl sarcosines, 2-alkylamino propionic acids and alkylamino acetic acids with in each case about 8 to 24 C atoms in the alkyl group. Typical examples of amphoteric or zwitterionic surfactants are alkyl betaines, alkylamido betaines, aminopropionates, amino glycinates, imidazolinium betaines and sulfobetaines.

Particularly preferred ampholytic surfactants are N-cocoalkyl aminopropionate, cocoacyl aminoethyl aminopropionate and C₁₂-C₁₈ acyl sarcosine.

Nonionic surfactants (Tnio) are, for example
addition products of 2 to 50 moles ethylene oxide and/or 0 to 5 moles propylene oxide to linear and branched fatty alcohols with 6 to 30 C atoms, fatty alcohol polyglycol ethers, fatty alcohol polypropylene glycol ethers and mixed fatty alcohol polyethers,
addition products of 2 to 50 moles ethylene oxide and/or 0 to 5 moles propylene oxide to linear and branched fatty acids with 6 to 30 C atoms, fatty acid polyglycol ethers, fatty acid polypropylene glycol ethers and mixed fatty acid polyethers,
addition products of 2 to 50 moles ethylene oxide and/or 0 to 5 moles propylene oxide to linear and branched alkylphenols with 8 to 15 C atoms in the alkyl group, alkylphenol polyglycol ethers, alkyl polypropylene glycol ethers, and mixed alkylphenol polyethers,
addition products of 2 to 50 moles ethylene oxide and/or 0 to 5 moles propylene oxide to linear and branched fatty alcohols with 8 to 30 C atoms, to fatty acids with 8 to 30 C atoms and to alkylphenols with 8 to 15 C atoms in the alkyl group, end-capped with a methyl or C₂-C₆ alkyl residue, such as for example the grades obtainable with the trade names Dehydrol® LS and Dehydrol® LT (Cognis),
C₁₂-C₃₀ fatty acid mono- and diesters of addition products of 1 to 30 moles ethylene oxide to glycerol,
addition products of 5 to 60 moles ethylene oxide to castor oil and hydrogenated castor oil,
polyol fatty acid esters, such as for example the commercial product Hydagen® HSP (Cognis) or Sovermol® grades (Cognis),
alkoxylated triglycerides,
alkoxylated fatty acid alkyl esters of the formula (Tnio-1) R¹CO—(OCH₂CHR²)_{w}OR³ (Tnio-1) in which R¹CO denotes a linear or branched, saturated and/or unsaturated acyl residue with 6 to 22 carbon atoms, R² denotes hydrogen or methyl, R³ denotes linear or branched alkyl residues with 1 to 4 carbon atoms and w denotes numbers from 1 to 20,
amine oxides,
hydroxy mixed ethers R¹O[CH₂CH(CH₃)O]ₓ(CH₂CHR²O)_{y}[CH₂CH(OH)R³]_{z} with R¹ denoting a linear or branched, saturated or unsaturated alkyl and/or alkenyl residue with 2 to 30 C atoms, R² denoting hydrogen, a methyl, ethyl, propyl or isopropyl residue, R³ denoting a linear or branched alkyl residue with 2 to 30 C atoms, x denoting 0 or a number from 1 to 20, Y denoting a number from 1 to 30 and z denoting the number 1, 2, 3, 4 or 5.
sorbitan fatty acid esters and addition products of ethylene oxide to sorbitan fatty acid esters such as, for example, polysorbates,
sugar fatty acid esters and addition products of ethylene oxide to sugar fatty acid esters,
addition products of ethylene oxide to fatty acid alkanolamides and fatty amines,
sugar surfactants of the alkyl and alkenyl oligoglycosides type,
sugar surfactants of the fatty acid N-alkyl polyhydroxyalkylamides type,
fatty acid amide polyglycol ethers, fatty amine polyglycol ethers,
mixed ethers or mixed formals and polysorbates.

Furthermore, for preference, at least one quaternary imidazoline compound, i.e. a compound having a positively charged imidazoline ring, is contained as cationic surfactant. Formula I illustrated below shows the structure of these compounds.

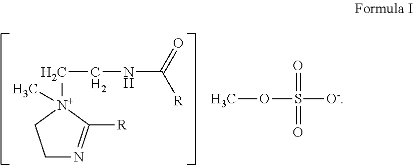

The residues R, independently of one another, each denote a saturated or unsaturated, linear or branched hydrocarbon residue with a chain length of 8 to 30 carbon atoms. The preferred compounds of formula I each contain the same hydrocarbon residue for R. The chain length of the residues R is preferably 12 carbon atoms. Particularly preferred are compounds with a chain length of at least 16 carbon atoms and most particularly preferably with at least 20 carbon atoms. A most particularly preferred compound of formula I has a chain length of 21 carbon atoms. A product of this chain length is known, for example, with the name Quaternium-91 or the trade names Crodazosoft® DBQ, which, in addition to Quaternium-91, also contains cetrimonium methosulfate and cetearyl alcohol, as well as Crodazosoft® SCQ, which, in addition to Quatemium-91, also contains PPG-3 benzyl ether myristate. In formula I, methosulfate is illustrated as the counter-ion. According to the invention, however, the halides such as chloride, fluoride and bromide, or also phosphates, are also included as counter-ions. Particular examples according to the invention are obtainable, for example, with the INCI names Quatemium-27, Quaternium-72, Quaternium-83 and Quaternium-91. The commercial products Crodazosoft® DBQ and Crodazosoft® SCQ, or Quaternium-91, are most preferably used.

The imidazolines of formula I are contained in the compositions according to the invention in amounts of 0.01 to 20 wt. %, preferably in amounts of 0.05 to 10 wt % and most particularly preferably in amounts of 0.1 to 7.5 wt. %. The best results of all are obtained with amounts of 0.1 to 5 wt. %, based in each case on the overall composition of the respective agent.

Furthermore, the following cationic surfactants according to the formula (Tkat-2) can be used. RCO—X—N⁺R¹R²R³ A⁻ (Tkat-2)

R here denotes a substituted or unsubstituted, branched or straight-chain alkyl or alkenyl residue with 11 to 35 carbon atoms in the chain,

X denotes —O— or —NR⁵—,
R¹ denotes an alkylene group with 2 to 6 C atoms, which can be unsubstituted or substituted, substitution with an —OH— or —NH— group being preferred if a substitution is present,
R², R³, each independently of one another, denote an alkyl or hydroxyalkyl group with 1 up to 6 C atoms in the chain, the chain being linear or branched,
R⁵ denotes hydrogen or a C₁ to C₆ straight-chain or branched, alkyl or alkenyl residue, which can also be substituted by a hydroxy group.

Within this class of structures, the compounds with one of the following structures are preferably used:

CH₃(CH₂)₂₀CONH(CH₂)₃—N⁺(CH₃)₂—CH₂CH₃A⁻ (Tkat-3)

CH₃(CH₂)₂₀CONH(CH₂)₃—N⁺(CH₃)₂—CH₂(CHOH)CH₂OHA⁻ (Tkat-4)

CH₃(CH₂)₂₀COOCH₂CHOHCH₂—N⁺(CH₃)₃A⁻ (Tkat-5)

CH₃(CH₂)₂₀CONH(CH₂)₃—N⁺(CH₃)₂—CH₂CH₂OHA⁻ (Tkat-6)

Examples of commercial products of this type are Schercoquat BAS, Lexquat AMG-BEO, Akypoquat 131 or Incroquat Behenyl HE.

Furthermore, esterquats according to the formula (Tkat1-2) can be used.

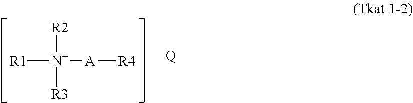

The residues R1, R2 and R3 are each independent of one another here and can be the same or different. The residues R1, R2 and R3 signify:
a branched or unbranched alkyl residue with 1 to 4 carbon atoms, which can contain at least one hydroxyl group, or
a saturated or unsaturated, branched or unbranched or a cyclic saturated or unsaturated alkyl residue with 6 to 30 carbon atoms, which can contain at least one hydroxyl group, or
an aryl or alkaryl residue, for example phenyl or benzyl,
the residue (-A-R4), with the proviso that no more than 2 of the residues R1, R2 or R3 can denote this residue.

The residue -(A-R4) is contained at least 1 to 3 times.

A here denotes:
1) —(CH2)n with n=1 to 20, preferably n=1 to 10 and particularly preferably n=1-5, or
2) —(CH2-CHR5-O)n- with n=1 to 200, preferably 1 to 100, particularly preferably 1 to 50 and particularly preferably 1 to 20, with R5 in the meaning of hydrogen, methyl or ethyl,
   and R4 denotes:
1) R6-O—CO—, wherein R6 is a saturated or unsaturated, branched or unbranched or a cyclic saturated or unsaturated alkyl residue with 6 to 30 carbon atoms, which can contain at least one hydroxy group and which can, in addition, optionally be oxyethylated with 1 to 100 ethylene oxide units and/or 1 to 100 propylene oxide units, or
2) R7-CO—, wherein R7 is a saturated or unsaturated, branched or unbranched or a cyclic saturated or unsaturated alkyl residue with 6 to 30 carbon atoms, which can contain at least one hydroxy group and which can, in addition, optionally be oxyethylated with 1 to 100 ethylene oxide units and/or 1 to 100 propylene oxide units,
   and Q denotes a physiologically acceptable organic or inorganic anion.

Products of this type are marketed for example with the trade names Rewoquat®, Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80, Dehyquart® F-30, Dehyquart® Rewoquat® WE18, Rewoquat® WE38 DPG and Stepantex® VS 90 are examples of these esterquats.

Other particularly preferred compounds of the formula (Tkat1-2) according to the invention are included in the formula (Tkat1-2.1), the cationic betaine esters.

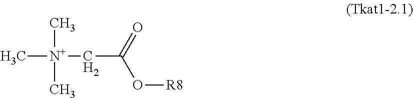

R8 corresponds in its meaning to R7.

As a further ingredient, monoalkyl trimethylammonium salts with a chain length of the alkyl residue of 16 to 24 carbon atoms can be contained.

These compounds have the structure illustrated in the formula (Tkat1-1),

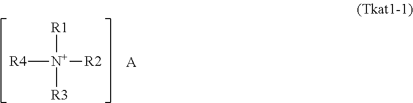

wherein R1, R2 and R3 each denote a methyl group and R4 denotes a saturated, branched or unbranched alkyl residue with a chain length of 16 to 24 carbon atoms. Examples of compounds of the formula (Tkat1-1) are cetyl trimethylammonium chloride, cetyl trimethylammonium bromide, cetyl trimethylammonium methosulfate, stearyl trimethylammonium chloride, behenyl trimethylammonium chloride, behenyl trimethylammonium bromide and behenyl trimethylammonium methosulfate.

In a particularly preferred embodiment of the invention, the agents according to the invention additionally contain at least one amine and/or cationized amine, in particular an amidoamine and/or a cationized amidoamine with the following structural formulae:

R¹—NH —(CH₂)ₙ—NR²R³ (Tkat7) and/or

R¹—NH —(CH₂)ₙ—NR²R³R⁴ (Tkat8)

wherein R1 is an acyl or alkyl residue with 6 to 30 C atoms, which can be branched or unbranched, saturated or unsaturated, and wherein the acyl residue and/or the alkyl residue can contain at least one OH group, and
R2, R3 and R4, each independently of one another, signify hydrogen or an alkyl residue with 1 to 4 C atoms, which can be the same or different, saturated or unsaturated, and
X⁻ signifies an anion and
n signifies an integer between 1 and 10.

A composition is preferred in which the amine and/or the quaternized amine according to general formulae (Tkat7) and/or (Tkat8) is an amidoamine and/or a quaternized amidoamine, wherein R1 signifies a branched or unbranched, saturated or unsaturated acyl residue with 6 to 30 C atoms, which can contain at least one OH group. A fatty acid residue from oils and waxes, in particular natural oils and waxes, is preferred here. Suitable examples of these are lanolin, beeswax or candellila wax.

Those amidoamines and/or quaternized amidoamines are also preferred in which R2, R3 and/or R4 in formulae (Tkat7) and/or (Tkat8) signify a residue according to the general formula CH₂CH₂OR5, wherein R5 can have the meaning of alkyl residues with 1 to 4 carbon atoms, hydroxyethyl or hydrogen. The preferred value of n in the general formulae (Tkat7) and/or (Tkat8) is an integer between 2 and 5.

Also preferred are amidoamines and/or quaternized amidoamines of the general formulae (Tkat7) and/or (Tkat8), in which the anion X⁻ is a halide ion or a compound of the general formula RSO₃⁻, wherein R has the meaning of saturated or unsaturated alkyl residues with 1 to 4 carbon atoms.

The alkyl residue with 1 to 4 carbon atoms of R2, R3 and R4 and/or the alkyl residue with 1 to 4 carbon atoms of RSO₃⁻ in the general formula (Tkat7) and/or (Tkat8) can contain at least one hydroxyl group.

The alkylamidoamines can both be present as such and can be converted by protonation in an appropriately acidic solution into a quaternary compound in the composition. The cationic alkylamidoamines are preferred according to the invention.

Examples of suitable amidoamines to be used according to the invention, which may optionally be quaternized, are, as amidoamines: Witcamine 100 (Witco, INCI name: Cocamidopropyl Dimethylamine), Incromine BB (Croda, INCI name: Behenamidopropyl Dimethylamine), Mackine 401 (McIntyre, INCI name: Isostearylamidopropyl Dimethylamine) and other Mackine grades, Adogen S18V (Witco, INCI name: Stearylamidopropyl Dimethylamine), and as permanent cationic aminoamines: Rewoquat RTM 50 (Witco Surfactants GmbH, INCI name: Ricinoleamidopropyltrimonium Methosulfate), Empigen CSC (Albright&Wilson, INCI name. Cocamidopropyltrimonium Chloride), Swanol Lanoquat DES-50 (Nikko, INCI name: Quaternium-33), Rewoquat UTM 50 (Witco Surfactants GmbH, Undecyleneamidopropyltrimonium Metho sulfate).

The anion of all the cationic compounds is selected from the physiologically acceptable anions. The halide ions fluoride, chloride, bromide, sulfate of the general formula RSO₃″, wherein R has the meaning of saturated or unsaturated alkyl residues with 1 to 4 carbon atoms, or anionic residues of organic acids, such as maleate, fumarate, oxalate, tartrate, citrate, lactate or acetate, may be mentioned as examples of these.

The above-mentioned cationic surfactants can be used individually or in any combinations with one another, with amounts between 0.01 and 20 wt. %, preferably in amounts of 0.01 to 10 wt. % and most particularly preferably in amounts of 0.1 to 7.5 wt. %, being contained. The best results of all are obtained with amounts of 0.1 to 5 wt. %, based in each case on the overall composition of the respective agent.

The surfactants (T) are used in amounts of 0.05-45 wt. %, preferably 0.1-30 wt. % and most particularly preferably of 0.5-25 wt. %, based on the total agent used according to the invention.

Emulsifiers that can be used according to the invention are, for example
addition products of 4 to 30 moles ethylene oxide and/or 0 to 5 moles propylene oxide to linear fatty alcohols with 8 to 22 C atoms, to fatty acids with 12 to 22 C atoms and to alkylphenols with 8 to 15 C atoms in the alkyl group,
C₁₂-C₂₂ fatty acid mono- and diesters of addition products of 1 to 30 moles ethylene oxide to polyols with 3 to 6 carbon atoms, in particular to glycerol,
ethylene oxide and polyglycerol addition products to methyl glucoside fatty acid esters, fatty acid alkanolamides and fatty acid glucamides,
C₈-C₂₂ alkyl mono- and oligoglycosides and the ethoxylated analogs thereof, with degrees of oligomerization of 1.1 to 5, in particular 1.2 to 2.0, and glucose as the sugar component, being preferred,
mixtures of alkyl(oligo)glucosides and fatty alcohols, for example the commercially available product Montanov®68,
addition products of 5 to 60 moles ethylene oxide to castor oil and hydrogenated castor oil,
partial esters of polyols with 3-6 carbon atoms with saturated fatty acids with 8 to 22 C atoms,
sterols, both from animal tissue (zoosterols, cholesterol, lanosterol) and from vegetable fats (phytosterols, ergosterol, stigmasterol, sitosterol) or from fungi and yeasts (mycosterols),
phospholipids (lecithins, phosphatidylcholine),
fatty acid esters of sugars and sugar alcohols, such as sorbitol,
   polyglycerols and polyglycerol derivatives, such as for example polyglycerol poly-12-hydroxystearate (commercial product Dehymuls® PGPH).

The agents according to the invention contain the emulsifiers preferably in amounts of 0.1-25 wt. %, in particular 0.5-15 wt. %, based on the total agent.

The cationic polymers can be homo- or copolymers, with the quaternary nitrogen groups being contained either in the polymer chain or preferably as a substituent on one or more of the monomers. The monomers containing ammonium groups can be copolymerized with non-cationic monomers. Suitable cationic monomers are unsaturated compounds capable of free-radical polymerization, which carry at least one cationic group, in particular ammonium-substituted vinyl monomers such as, for example, trialkyl methacryloxy alkylammonium, trialkyl acryloxy alkylammonium, dialkyldiallylammonium and quaternary vinylammonium monomers with cyclic, cationic nitrogen-containing groups, such as pyridinium, imidazolium or quaternary pyrrolidones, e.g. alkyl vinyl imidazolium, alkyl vinyl pyridinium, or alkyl vinylpyrrolidone salts. The alkyl groups of these monomers are preferably low alkyl groups such as, for example, C1 to C7 alkyl groups, particularly preferably C1 to C3 alkyl groups.

The monomers containing ammonium groups can be copolymerized with non-cationic monomers. Suitable comonomers are, for example, acrylamide, methacrylamide; alkyl- and dialkylacrylamide, alkyl- and dialkylmethacrylamide, alkyl acrylate, alkyl methacrylate, vinyl caprolactone, vinyl caprolactam, vinylpyrrolidone, vinyl esters, e.g. vinyl acetate, vinyl alcohol, propylene glycol or ethylene glycol, the alkyl groups of these monomers preferably being C1 to C7 alkyl groups, particularly preferably C1 to C3 alkyl groups.

Suitable polymers with quaternary amine groups are, for example, the polymers described in the CTFA Cosmetic Ingredient Dictionary with the names Polyquaternium, such as methyl vinylimidazolium chloride/vinylpyrrolidone copolymer (Polyquaternium-16) or quaternized vinylpyrrolidone/dimethylaminoethyl methacrylate copolymer (Polyquatemium-11).

Of the cationic polymers that can be contained in the agent according to the invention, for example vinylpyrrolidone/dimethylaminoethyl methacrylate methosulfate copolymer, which is marketed with the trade names Gafquat® 755 N and Gafquat® 734 by Gaf Co., USA and of which Gafquat® 734 is particularly preferred, are suitable. Other cationic polymers are, for example, the copolymer of polyvinylpyrrolidone and imidazolimine methochloride marketed by BASF, Germany with the trade name Luviquat® HM 550, the terpolymer of dimethyldiallylammonium chloride, sodium acrylate and acrylamide marketed by Calgon/USA with the trade name Merquat® Plus 3300 and the vinylpyrrolidone/methacrylamidopropyl trimethylammonium chloride copolymer marketed by ISP with the trade name Gafquat® HS 100.

Homopolymers of the general formula (P1),

—{CH₂—[CR¹COO—(CH₂)ₘN⁺R²R³R⁴]}ₙX⁻,

in which R¹=—H or —CH₃, R², R³ and R⁴, independently of one another, are selected from C₁₋₄ alkyl, alkenyl or hydroxyalkyl groups, m=1, 2, 3 or 4, n is a natural number and X⁻ is a physiologically acceptable organic or inorganic anion. In the context of these polymers, those for which at least one of the following conditions applies are preferred according to the invention: R¹ denotes a methyl group, R², R³ and R⁴ denote methyl groups, m has the value of 2.

Suitable as physiologically acceptable counter-ions X− are, for example, halide ions, sulfate ions, phosphate ions, methosulfate ions and organic ions such as lactate, citrate, tartrate and acetate ions. Preferred are halide ions, in particular chloride.

A particularly suitable homopolymer is the optionally crosslinked poly(methacryloyloxyethyl trimethylammonium chloride) with the INCI name Polyquaternium-37. Products of this type are commercially available, for example, with the names Rheocare® CTH (Cosmetic Rheologies) and Synthalen® CR (3V Sigma).

The homopolymer is preferably used in the form of a non-aqueous polymer dispersion. Polymer dispersions of this type are commercially available with the names Salcare® SC 95 and Salcare® SC 96.

A preferred copolymer according to the invention is the crosslinked acrylamide-methacryloyloxyethyl trimethylammonium chloride copolymer. Copolymers of this type are commercially available with the name Salcare® SC 92.

Suitable cationic polymers that are derived from natural polymers are cationic derivatives of polysaccharides, for example cationic derivatives of cellulose, starch or guar. Chitosan and chitosan derivatives are also suitable. Cationic polysaccharides have the general formula (P-3) G-O—B—N+RₐR_{b}R_{c}X⁻

G is an anhydroglucose residue, for example starch or cellulose anhydroglucose;
B is a group of divalent compounds, for example alkylene, oxyalkylene, polyoxyalkylene or hydroxyalkylene;
Rₐ, R_{b} and R_{c} are, independently of one another, alkyl, aryl, alkylaryl, arylalkyl, alkoxyalkyl or alkoxyaryl, each with up to 18 C atoms, the total number of C atoms in Rₐ, R_{b} and R_{c} preferably being no more than 20;
X⁻ is a conventional counter-anion and is preferably chloride.

A cationic cellulose is marketed by Amerchol with the name Polymer JR® 400 and has the INCI name Polyquaternium-10. Another cationic cellulose has the INCI name Polyquaternium-24 and is marketed by Amerchol with the trade name Polymer LM-200. Other commercial products are the compounds Celquat® H 100, Celquat® and L 200. The above commercial products are preferred cationic celluloses.

Suitable cationic guar derivatives are marketed with the trade name Jaguar® and have the INCI name Guar Hydroxypropyltrimonium Chloride. Furthermore, particularly suitable cationic guar derivatives are also commercially available from Hercules with the name N-Hance®. Other cationic guar derivatives are marketed by Cognis with the name Cosmedia®. A preferred cationic guar derivative is the commercial product AquaCat® from Hercules. This raw material is an already pre-dissolved cationic guar derivative.

A suitable chitosan is marketed, for example, by Kyowa Oil & Fat, Japan, with the trade name Flonac®. A preferred chitosan salt is chitosonium pyrrolidone carboxylate, which is marketed, for example, by Amerchol USA with the name Kytamer® PC. Other chitosan derivatives are readily available commercially with the trade names Hydagen® CMF, Hydagen® HCMF and Chitolam® NB/101.

Other preferred cationic polymers are, for example
cationic alkyl polyglycosides,
cationized honey, for example the commercial product Honeyquat® 50,
polymeric dimethyldiallylammonium salts and copolymers thereof with esters and amides of acrylic acid and methacrylic acid. The products commercially available with the names Merquat®100 (poly(dimethyldiallylammonium chloride)) and Merquat®550 (dimethyldiallylammonium chloride-acrylamide copolymer) are examples of these cationic polymers,
vinylpyrrolidone-vinylimidazolium methochloride copolymers, as made available with the names Luviquat® FC 370, FC 550, FC 905 and HM 552,
quaternized polyvinyl alcohol,
and the polymers known with the names Polyquaternium 2, Polyquaternium 17, Polyquaternium 18 and Polyquaternium 27 with quaternary nitrogen atoms in the main polymer chain,
   vinylpyrrolidone-vinylcaprolactam-acrylate terpolymers, as are offered commercially with acrylic esters and acrylamides as the third monomer building block, for example with the name Aquaflex® SF 40.

According to the invention, it is also possible to use the copolymers of vinylpyrrolidone, as are available as the commercial products Copolymer 845 (manufacturer: ISP), Gaffix® VC 713 (manufacturer: ISP), Gafquat®ASCP 1011, Gafquat®HS 110, Luviquat®8155 and Luviquat® MS 370.

The cationic polymers also include cationized protein hydrolyzates, in which case the basic protein hydrolyzate can originate from animals, for example from collagen, milk or keratin, from plants, for example from wheat, maize, rice, potatoes, soybean or almonds, from marine life forms, for example from fish collagen or algae, or biotechnologically obtained protein hydrolyzates. The commercially available products mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702) should be mentioned as typical examples of the cationic protein hydrolyzates and derivatives according to the invention.

The cationic polymers are contained in the compositions according to the invention preferably in amounts of 0.01 to 10 wt. %, based on the total agent. Amounts of 0.05 to 5 wt. % are particularly preferred.

Furthermore, it is possible to use amphoteric polymers as polymers. The term amphoteric polymers comprises both those polymers that contain both free amino groups and free —COOH— or SO₃H groups in the molecule and are capable of forming internal salts, and zwitterionic polymers that contain quaternary ammonium groups and —COO⁻ or —SO₃⁻ groups in the molecule, and those polymers that contain —COOH— or SO₃H groups and quaternary ammonium groups.

Preferred amphoteric and/or cationic polymers according to the invention are those polymers in which a cationic group is derived from at least one of the following monomers:
monomers with quaternary ammonium groups of the general formula (Mono1),

R¹—CH═CR²—CO—Z—(CₙH2ₙ)—N⁽⁺⁾R²R³R⁴ (Mono1)
in which R¹ and R², independently of one another, denote hydrogen or a methyl group and R³, R⁴ and R⁵, independently of one another, denote alkyl groups with 1 to 4 carbon atoms, Z is an NH group or an oxygen atom, n is an integer from 2 to 5 and A⁽⁻⁾ is the anion of an organic or inorganic acid,
(ii) monomers with quaternary ammonium groups of the general formula (Mono2),

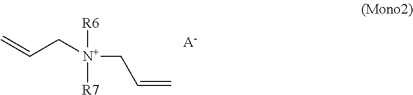

wherein R⁶ and R⁷, independently of one another, denote a (C₁ to C₄)alkyl group, in particular a methyl group, and
A⁻ is the anion of an organic or inorganic acid,
(iii) monomeric carboxylic acids of the general formula (Mono3),

R⁸—CH═CR⁹—COOH (Mono3)

in which R⁸ and R⁹, independently of one another, are hydrogen or methyl groups.

Particularly preferred are those polymers in which monomers of type (i) are used, in which R³, R⁴ and R⁵ are methyl groups, Z is an NH group and A⁽⁻⁾ is a halide, methoxy sulfate or ethoxy sulfate ion; acrylamidopropyl trimethyl-ammonium chloride is a particularly preferred monomer (i). As monomer (ii) for the above polymers, acrylic acid is preferably used.

Particularly preferred amphoteric polymers are copolymers of at least one monomer (Mono1) or (Mono2) with the monomer (Mono3), in particular copolymers of the monomers (Mono2) and (Mono3). Most particularly preferably used amphoteric polymers according to the invention are copolymers of diallyl dimethylammonium chloride and acrylic acid. These copolymers are marketed with the INCI name Polyquaternium-22, inter alia with the trade name Merquat® 280 (Nalco).

In addition, the amphoteric polymers according to the invention, as well as a monomer (Mono1) or (Mono2) and a monomer (Mono3), can additionally contain a monomer (Mono4)
(iv) monomeric carboxylic acid amides of the general formula (Mono4),

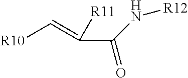

in which R¹⁰ and R¹¹, independently of one another, are hydrogen or methyl groups and R¹² denotes a hydrogen atom or a (C₁ to C₈)alkyl group.

Most particularly preferably used amphoteric polymers according to the invention based on a comonomer (Mono4) are terpolymers of diallyl dimethylammonium chloride, acrylamide and acrylic acid. These copolymers are marketed with the INCI name Polyquaternium-39, inter alia with the trade name Merquat® Plus 3330 (Nalco).

The amphoteric polymers can generally be used according to the invention either directly or in the form of a salt, which is obtained by neutralization of the polymers, for example with an alkali hydroxide.

The amphoteric polymers are contained in the agents according to the invention preferably in amounts of 0.01 to 10 wt. %, based on the total agent. Amounts of 0.01 to 5 wt. % are particularly preferred.

The anionic polymers are anionic polymers that have carboxylate and/or sulfonate groups. Examples of anionic monomers of which these polymers can consist are acrylic acid, methacrylic acid, crotonic acid, maleic anhydride and 2-acrylamido-2-methylpropanesulfonic acid. The acid groups here may be present entirely or partially as a sodium, potassium, ammonium, mono- or triethanolammonium salt. Preferred monomers are 2-acrylamido-2-methylpropanesulfonic acid and acrylic acid.

Anionic polymers which contain 2-acrylamido-2-methylpropanesulfonic acid as sole monomer or comonomer, wherein the sulfonic acid group may be present entirely or partially as a sodium, potassium, ammonium, mono- or triethanolammonium salt, have proved to be most particularly effective.

Particularly preferred is the homopolymer of 2-acrylamido-2-methylpropanesulfonic acid, which is commercially available for example with the name Rheothik® 11-80.

Within this embodiment it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic esters, methacrylic esters, vinylpyrrolidone, vinyl ethers and vinyl esters.

Preferred anionic copolymers are acrylic acid-acrylamide copolymers and, in particular, polyacrylamide copolymers with monomers containing sulfonic acid groups. One such polymer is contained in the commercial product Sepigel® 305 from SEPPIC.

The sodium acryloyldimethyl taurate copolymers marketed with the name Simulgel®600 as a compound with isohexadecane and Polysorbate 80 have also proved particularly effective according to the invention.

Likewise preferred anionic homopolymers are uncrosslinked and crosslinked polyacrylic acids. Allyl ethers of pentaerythritol, of sucrose and of propylene can be preferred crosslinking agents here. Compounds of this type are commercially available for example with the trademark Carbopol®.

Copolymers of maleic anhydride and methyl vinyl ether, in particular those with crosslinks, are likewise color-preserving polymers. A maleic acid/methyl vinyl ether copolymer crosslinked with 1,9-decadiene is commercially available with the trade name Stabileze® QM.

The anionic polymers are contained in the agents according to the invention preferably in amounts of 0.05 to 10 wt. %, based on the total agent. Amounts of 0.1 to 5 wt. % are particularly preferred.

A most particularly preferred polyurethane according to the invention is on the market with the trade name Luviset® PUR (BASF).

The agents according to the invention can contain nonionogenic polymers in a further embodiment.

Suitable nonionogenic polymers are, for example:
vinylpyrrolidone/vinyl ester copolymers, as are marketed for example with the trademark Luviskol® (BASF). Luviskol® VA 64 and Luviskol® VA 73, both vinylpyrrolidone/vinyl acetate copolymers, are likewise preferred nonionic polymers.
cellulose ethers, such as hydroxypropyl cellulose, hydroxyethyl cellulose and methyl hydroxypropyl cellulose, as are marketed for example with the trademarks Culminal® and Benecel® (AQUALON) and Natrosol® grades (Hercules).
starch and derivatives thereof, in particular starch ethers, for example Structure® XL (National Starch), a multifunctional, salt-tolerant starch;
shellac,
polyvinylpyrrolidones, as marketed for example with the name Luviskol® (BASF).

The nonionic polymers are contained in the compositions according to the invention preferably in amounts of from 0.05 to 10 wt. %, based on the total agent. Amounts of 0.1 to 5 wt. % are particularly preferred.

It is also possible according to the invention that the preparations used contain more than one, in particular two, different polymers with the same charge and/or one ionic and one amphoteric and/or nonionic polymer.

The polymers (P) are contained in the compositions used according to the invention preferably in amounts of 0.01 to 30 wt. %, based on the total composition. Amounts of 0.01 to 25, in particular of 0.01 to 15 wt. %, are particularly preferred.

In addition to the essential components, the agents according to the invention preferably contain as a further ingredient at least one silicone polymer selected from the group of the dimethiconols and/or the group of the amino-functional silicones and/or the group of the dimethicones and/or the group of the cyclomethicones. These ingredients are described in the following.

The dimethicones according to the invention can be linear and branched and cyclic or cyclic and branched. Linear dimethicones can be represented by the following structural formula (Si1):

(SiR¹₃)—O—(SiR²₂—O—(SiR¹₃) (Si1)

Branched dimethicones can be represented by the structural formula (Si1.1):

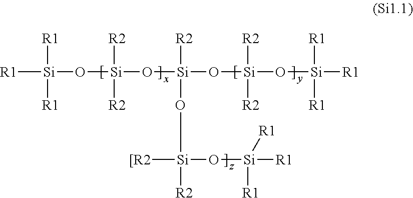

The residues R¹ and R², independently of one another, each represent hydrogen, a methyl residue, a C₂ to C₃₀ linear, saturated or unsaturated hydrocarbon residue, a phenyl residue and/or an aryl residue. The numbers x, y and z are integers and, each independently of one another, run from 0 to 50,000. The molecular weights of the dimethicones are between 1000 D and 10,000,000 D. The viscosities are between 100 and 10,000,000 cPs measured at 25° C. with the aid of a glass capillary viscometer in accordance with the Dow Corning Corporate Test Method CTM 0004 of 20 Jul. 1970. Preferred viscosities are between 1000 and 5,000,000 cPs; more particularly preferred viscosities are between 10,000 and 3,000,000 cPs. The most preferred range is between 50,000 and 2,000,000 cPs. Extremely preferred are viscosities around the range of about 60,000 cPs. Reference is made here by way of example to the product “Dow Corning 200 with 60000 cSt”.

Particularly preferred cosmetic or dermatological preparations according to the invention are characterized in that they contain at least one silicone of the formula (Si1.2)

(CH₃)₃Si[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are contained in the compositions according to the invention in amounts of 0.01 to 10 wt. %, preferably 0.01 to 8 wt. %, particularly preferably 0.1 to 7.5 wt. % and in particular 0.1 to 5 wt. %, based on the total composition.

Particularly preferred agents according to the invention contain one or more amino-functional silicones. These silicones can be described e.g. by the formula (Si-2)

M(RₐQ_{b}SiO_{(4-a-b)/2})ₓ(R_{c}SiO_{(4-c)/2})_{y}M (Si-2)

wherein in the above formula
R is a hydrocarbon or a hydrocarbon residue with 1 to about 6 carbon atoms,
Q is a polar residue of the general formula —R¹HZ,
   wherein
   R¹ is a divalent linking group, which is bonded to hydrogen and the residue Z, composed of carbon and hydrogen atoms, carbon, hydrogen and oxygen atoms or carbon, hydrogen and nitrogen atoms, and
   Z is an organic, amino-functional residue, which contains at least one amino-functional group;
a assumes values in the range from about 0 to about 2,
b assumes values in the range from about 1 to about 3,
a+b is less than or equal to 3, and
c is a number in the range from about 1 to about 3, and
x is a number in the range from 1 to about 2,000, preferably from about 3 to about 50 and most preferably from about 3 to about 25, and
y is a number in the range from about 20 to about 10,000, preferably from about 125 to about 10,000 and most preferably from about 150 to about 1,000, and
M is a suitable silicone end group, as is known in the prior art, preferably trimethylsiloxy.

Z according to formula (Si-2) is an organic, amino-functional residue containing at least one functional amino group. One possible formula for said Z is NH(CH₂)_{z}NH₂, wherein z is an integer greater than or equal to 1. Another possible formula for said Z is —NH(CH₂)_{z}(CH₂)_{zz}NH, wherein both z and zz, independently of one another, are an integer greater than or equal to 1, wherein this structure comprises diamino ring structures, such as piperazinyl. Said Z is most preferably an —NHCH₂CH₂NH₂— residue. Another possible formula for said Z is —N(CH₂)_{z}(CH₂)_{zz}NX₂ or —NX₂, wherein each X of X₂ is independently selected from the group consisting of hydrogen and alkyl groups with 1 to 12 carbon atoms, and zz is 0.

Q according to formula (Si-2) is most preferably a polar amino-functional residue of the formula —CH₂CH₂CH₂NHCH₂CH₂NH₂.

In the formula (Si-2), a assumes values in the range of 0 to 2, b assumes values in the range of 2 to 3, a+b is less than or equal to 3 and c is a number in the range of 1 to 3. Suitable according to the invention are cationic silicone oils such as, for example, the commercially available products Dow Corning (DC) 929 Emulsion, DC 2-2078, DC 5-7113, SM-2059 (General Electric) and SLM-55067 (Wacker).

Particularly preferred agents according to the invention are characterized in that they contain at least one amino-functional silicone of the formula (Si3-a)

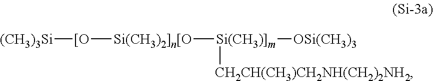

wherein m and n are numbers, the sum (m+n) of which is between 1 and 2000, preferably between 50 and 150, wherein n preferably assumes values from 0 to 1999 and in particular from 49 to 149, and m preferably assumes values from 1 to 2000, in particular from 1 to 10.

These silicones are designated as trimethylsilylamodimethicones according to the INCI Declaration and are obtainable, for example, with the name Q2-7224 (manufacturer: Dow Corning; a stabilized trimethylsilylamodimethicone).

Particularly preferred agents according to the invention are also those containing at least one amino-functional silicone of the formula (Si-3b)

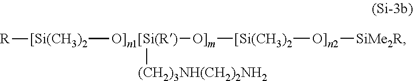

wherein
R denotes —OH, an (optionally ethoxylated and/or propoxylated) (C₁ to C₂₀)alkoxy group or a —CH₃ group,
R′ denotes —OH, a (C₁ to C₂₀)alkoxy group or a —CH₃ group and
m, n1 and n2 are numbers, the sum (m+n1+n2) of which is between 1 and 2000, preferably between 50 and 150, the sum (n1+n2) preferably assuming values from 0 to 1999 and in particular from 49 to 149, and m preferably assuming values from 1 to 2000, in particular from 1 to 10.

These silicones are designated according to the INCI Declaration as amodimethicones, or as functionalized amodimethicones, such as for example Bis(C13-15 Alkoxy) PG Amodimethicone (obtainable, for example, as the commercial product: DC 8500 from Dow Corning) or Trideceth-9 PG-Amodimethicone (obtainable, for example, as the commercial product Silcare Silicone SEA from Clariant). Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

wherein the residues R1 to R6, independently of one another, signify C1 to C22 alkyl residues, which can contain hydroxy groups and wherein preferably at least one of the residues has at least 8 C atoms and the remaining residues have 1 to 4 C atoms,
the residues R7 to R12, independently of one another, are the same or different and signify C1 to C10 alkyl or phenyl,
A signifies a group of divalent organic compounds,
n is a number from 0 to 200, preferably from 10 to 120, particularly preferably from 10 to 40,
and X⁻ is an anion.

The group of divalent compounds is preferably a C1 to C12 alkylene or alkoxyalkylene group, which can be substituted with one or more hydroxyl groups. The group —(CH₂)₃—O—CH₂—CH(OH)—CH₂— is particularly preferred.

The anion X⁻ can be a halide ion, an acetate, an organic carboxylate or a compound of the general formula RSO₃⁻, wherein R has the meaning of C1 to C4 alkyl residues.

A preferred diquaternary silicone has the general formula (Si3d)

[RN⁺Me₂-A-(SiMe₂O)ₙ—SiMe₂-A-N⁺Me₂R]₂CH₃COO⁻ (Si3d),

wherein A is the group —(CH₂)₃—O—CH₂—CH(OH)—CH₂—,
R is an alkyl residue with at least 8 C atoms and n is a number from 10 to 120.

Suitable silicone polymers with two terminal, quaternary ammonium groups are known with the INCI name Quaternium-80. These are dimethylsiloxanes with two terminal trialkylammonium groups. Diquaternary polydimethylsiloxanes of this type are marketed by Evonik with the trade names Abil® Quat 3270, 3272 and 3474.

Preferred cosmetic or dermatological preparations according to the invention are characterized in that, based on their weight, they contain 0.01 to 10 wt. %, preferably 0.01 to 8 wt. %, particularly preferably 0.1 to 7.5 wt. % and in particular 0.2 to 5 wt. % amino-functional silicone(s) and/or diquaternary silicone.

As silicone, the compositions according to the invention can contain at least one polyammonium-polysiloxane compound. The polyammonium-polysiloxane compounds can be obtained for example with the trade name Baysilone® from GE Bayer Silicones. The products with the names Baysilone TP 391 1, SME 253 and SFE 839 are preferred here. Most particularly preferred is the use of Baysilone TP 3911 as active component of the compositions according to the invention. The polyammonium-polysiloxane compounds are used in the compositions according to the invention in an amount of 0.01 to 10 wt. %, preferably 0.01 to 7.5, particularly preferably 0.01 to 5.0 wt. %, most particularly preferably from 0.05 to 2.5 wt. %, based in each case on the overall composition.

The cyclic dimethicones referred to according to INCI as Cyclomethicones can also be used with preference according to the invention. Cosmetic or dermatological preparations according to the invention are preferred here which contain at least one silicone of the formula (Si-4)

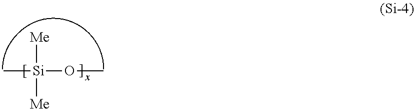

in which x denotes a number from 3 to 200, preferably from 3 to 10, more preferably from 3 to 7 and in particular 3, 4, 5 or 6.

Likewise, preferred agents according to the invention are characterized in that they contain at least one silicone of the formula (Si-5)

R₃Si—[O—SiR₂]ₓ—(CH₂)ₙ—[O—SiR₂]_{y}—O—SiR₃ (Si-5),

in which R denotes the same or different residues from the group —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As further silicones in addition to the dimethicones, dimethiconols, amodimethicones and/or cyclomethicones according to the invention, water-soluble silicones can also be contained in the compositions according to the invention.

Suitable hydrophilic silicones are selected for example from the compounds of the formulae (Si-6) and/or (Si-7). Particularly preferred water-soluble silicone-based surfactants are selected from the group of the dimethicone copolyols, which are preferably alkoxylated, particularly polyethoxylated or polypropoxylated.

Dimethicone copolyols according to the invention are understood to be preferably polyoxyalkylene-modified dimethyl polysiloxanes of the general formulae (Si-6) or (Si-7):

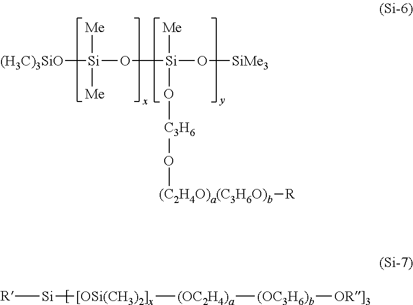

wherein the residue R denotes a hydrogen atom, an alkyl group with 1 to 12 C atoms, an alkoxy group with 1 to 12 C atoms or a hydroxyl group, the residues R′ and R″ signify alkyl groups with 1 to 12 C atoms, x denotes an integer from 1 to 100, preferably from 20 to 30, y denotes an integer from 1 to 20, preferably from 2 to 10, and a and b denote integers from 0 to 50, preferably from 10 to 30.

Particularly preferred dimethicone copolyols within the meaning of the invention are, for example, the products marketed commercially with the trade name SILWET (Union Carbide Corporation) and DOW CORNING. Particularly preferred dimethicone copolyols according to the invention are Dow Corning 190 and Dow Corning 193.

The dimethicone copolyols are contained in the compositions according to the invention in amounts of 0.01 to 10 wt. %, preferably 0.01 to 8 wt. %, particularly preferably 0.1 to 7.5 wt. % and in particular 0.1 to 5 wt. % of dimethicone copolyol, based on the composition.

Finally, the silicone compounds are understood to be the dimethiconols (Si8). The dimethiconols according to the invention can be both linear and branched as well as cyclic or cyclic and branched. Linear dimethiconols can be represented by the following structural formula (Si8-I):

(SiOHR¹₂)—O—(SiR²₂—O—)ₓ—(SiOHR¹₂) (Si8-I)

Branched dimethiconols can be represented by the structural formula (Si8-II):

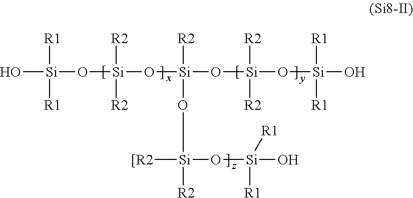

The residues R¹ and R², independently of one another, each denote hydrogen, a methyl residue, a C2 to C30 linear, saturated or unsaturated hydrocarbon residue, a phenyl residue and/or an aryl residue. The numbers x, y and z are integers and, each independently of one another, run from 0 to 50,000. The molecular weights of the dimethiconols are between 1000 D and 10,000,000 D. The viscosities are between 100 and 10,000,000 cPs measured at 25° C. with the aid of a glass capillary viscometer in accordance with the Dow Corning Corporate Test Method CTM 0004 of 20 Jul. 1970. Preferred viscosities are between 1000 and 5,000,000 cPs; more particularly preferred viscosities are between 10,000 and 3,000,000 cPs. The most preferred range is between 50,000 and 2,000,000 cPs.

The following commercial products are mentioned as examples of these products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (all four of the above GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all of the above Wacker-Chemie GmbH).

The dimethiconols (Si8) are contained in the compositions according to the invention in amounts of 0.01 to 10 wt. %, preferably 0.01 to 8 wt. %, particularly preferably 0.1 to 7.5 wt. % and in particular 0.1 to 5 wt. % of dimethiconol, based on the composition.

The compositions according to the invention particularly preferably contain fatty substances (Fat) as an additional active substance. Fatty substances (Fat) are understood to be fatty acids, fatty alcohols, natural and synthetic waxes, which can be present both in solid form and as a liquid in aqueous dispersion, and natural and synthetic cosmetic oil components.

As fatty acids (Fatac), it is possible to use linear and/or branched, saturated and/or unsaturated fatty acids with 6-30 carbon atoms. Fatty acids with 10-22 carbon atoms are preferred. Among these, the following should be mentioned, for example: the isostearic acids, such as the commercial products Emersol® 871 and Emersol® 875, and isopalmitic acids, such as the commercial product Edenor® IP 95, as well as all other fatty acids marketed with the trade names Edenor® (Cognis). Other typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. The fatty acid blends obtainable from coconut oil or palm oil are usually particularly preferred; the use of stearic acid is, in general, especially preferred.

The amount used in this case is 0.1-15 wt. %, based on the total agent. The amount is preferably 0.5-10 wt. %, and amounts of 1-5 wt. % can be most particularly advantageous.

As fatty alcohols (Fatal), it is possible to use saturated, mono- or polyunsaturated, branched or unbranched fatty alcohols with C₆-C₃₀, preferably C₁₀-C₂₂ and most particularly preferably C₁₂-C₂₂ carbon atoms. Within the meaning of the invention it is possible to use, for example, decanol, octanol, octenol, dodecenol, decenol, octadienol, dodecadienol, decadienol, cetyl alcohol, erucyl alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as Guerbet alcohols thereof, this list being intended to have an exemplary and not a limiting nature. The fatty alcohols preferably originate from natural fatty acids, however, in which case it can usually be assumed that they are obtained from the esters of the fatty acids by reduction. It is likewise possible according to the invention to use those fatty alcohol blends that represent a mixture of different fatty alcohols. Such substances can be purchased, for example, with the names Stenol®, e.g. Stenol® 1618, or Lanette®, e.g. Lanette® O, or Lorol®, e.g. Lorol®C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible according to the invention to use wool wax alcohols, as can be purchased, for example, with the names Corona®, White Swan®, Coronet® or Fluilan®. The fatty alcohols are used in amounts of 0.1-30 wt. %, based on the total preparation, preferably in amounts of 0.1-20 wt. %.

As natural or synthetic waxes (Fatwax), it is possible according to the invention to use solid paraffins or isoparaffins, carnauba waxes, beeswaxes, candellila waxes, ozokerites, ceresin, cetaceum, sunflower wax, fruit waxes, such as for example apple wax or citrus wax, microwaxes of PE or PP. Waxes of this type are obtainable, for example, through Kahl & Co., Trittau.

The amount used is 0.1-50 wt. %, based on the total agent, preferably 0.1-20 wt. % and particularly preferably 0.1-15 wt. %, based on the total agent.

The total amount of oil and fat components in the agents according to the invention is usually 0.5-75 wt. %, based on the total agent. Amounts of 0.5-35 wt. % are preferred according to the invention.

Protein hydrolyzates and/or the derivatives thereof (P) are another synergistic active substance according to the invention in the compositions according to the invention with the active substance complex according to the invention.

According to the invention, it is possible to use protein hydrolyzates of both vegetable and animal or marine or synthetic origin.

Animal protein hydrolyzates are, for example, elastin, collagen, keratin, silk and milk protein hydrolyzates, which may also be present in the form of salts. These products are marketed, for example, with the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Furthermore, preferred vegetable protein hydrolyzates according to the invention are, for example, soybean, almond, pea, moring a, potato and wheat protein hydrolyzates. These products are obtainable, for example, with the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Sérobiologiques.

Other preferred protein hydrolyzates according to the invention are of maritime origin. These include, for example, collagen hydrolyzates from fish or algae and protein hydrolyzates from mussels or pearl hydrolyzates. Examples of pearl extracts according to the invention are the commercial products Pearl Protein Extract BG® or Crodarom® Pearl.

The protein hydrolyzates (P) are contained in the compositions in concentrations of 0.001 wt. % up to 20 wt. %, preferably of 0.05 wt. % up to 15 wt. % and most particularly preferably in amounts of 0.05 wt. % up to 5 wt. %.

The action of the compositions according to the invention can furthermore be increased by a 2-pyrrolidinone-5-carboxylic acid and the derivatives thereof (J). The sodium, potassium, calcium, magnesium or ammonium salts, in which the ammonium ion has one to three C₁ to C₄ alkyl groups in addition to hydrogen, are preferred. The sodium salt is most particularly preferred. The amounts used in the agents according to the invention are 0.05 to 10 wt. %, based on the total agent, particularly preferably 0.1 to 5, and in particular 0.1 to 3 wt. %.

Vitamins, provitamins or vitamin precursors are another preferred group of ingredients of the compositions according to the invention with the active substance complex according to the invention. Vitamins, pro-vitamins and vitamin precursors that are allocated to the groups A, B, C, E, F and H are particularly preferred here.

The group of substances referred to as vitamin A includes retinol (vitamin A₁) and 3,4-didehydroretinol (vitamin A₂). β-Carotene is the provitamin of retinol. Suitable as vitamin A component according to the invention are, for example, vitamin A acid and esters thereof, vitamin A aldehyde and vitamin A alcohol and esters thereof, such as palmitate and acetate. The agents according to the invention contain the vitamin A component preferably in amounts of 0.05-1 wt. %, based on the total preparation.

Vitamin C (ascorbic acid). Vitamin C is used in the agents according to the invention preferably in amounts of 0.1 to 3 wt. %, based on the total agent. Use in the form of the palmitic acid ester, the glucosides or phosphates may be preferred. Use in combination with tocopherols may likewise be preferred.

Vitamin E (tocopherols, in particular α-tocopherol). Tocopherol and its derivatives, including in particular the esters, such as the acetate, nicotinate, phosphate and succinate, are contained in the agents according to the invention preferably in amounts of 0.05-1 wt. %, based on the total agent.

Vitamin F The term “vitamin F” is usually understood to mean essential fatty acids, in particular linoleic acid, linolenic acid and arachidonic acid.

Vitamin H. The compound (3aS,4S,6aR)-2-oxohexahydrothienol[3,4-d]-imidazole-4-valeric acid is referred to as vitamin H, but its trivial name biotin has now become accepted. Biotin is contained in the agents according to the invention preferably in amounts of 0.0001 to 1.0 wt. %, in particular in amounts of 0.001 to 0.01 wt. %.

The compositions according to the invention preferably contain as further vitamins, in addition to a B series vitamin, vitamins, provitamins and vitamin precursors from the groups A, E and H.

A particularly preferred group of ingredients in the cosmetic compositions according to the invention comprises the betaines mentioned below: carnitine, carnitine tartrate, carnitine magnesium citrate, acetyl carnitine, betalains, 1,1-dimethylproline, choline, choline chloride, choline bitartrate, choline dihydrogen citrate and the compound N,N,N-trimethylglycine, referred to in the literature as betaine.

Carnitine, histidine, choline and betaine are preferably used. In a particularly preferred embodiment of the invention, L-carnitine tartrate is used as an active substance.

One particularly essential ingredient is taurine and/or a derivative of taurine. Taurine is understood exclusively as 2-aminoethanesulfonic acid and a derivative as the derivatives of taurine mentioned explicitly. The derivatives of taurine are understood as N-monomethyl taurine, N,N-dimethyl taurine, taurine lysylate, taurine tartrate, taurine ornithate, lysyl taurine and ornithyl taurine. Other taurine derivatives within the meaning of the present invention are taurocholic acid and hypotaurine.

Particularly preferred are agents according to the invention which—based on their weight—contain 0.0001 to 10.0 wt. %, preferably 0.0005 to 5.0 wt. %, particularly preferably 0.001 to 2.0 wt. % and in particular 0.001 to 1.0 wt. % taurine and/or a derivative of taurine.

In another preferred embodiment according to the invention, the compositions according to the invention contain bio-quinones. In the agents according to the invention, suitable bio-quinones are understood to be one or more ubiquinone(s) and/or plastoquinone(s). The preferred ubiquinones according to the invention have the following formula:

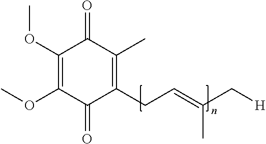

with n=6, 7, 8, 9 or 10.

Coenzyme Q-10 is most preferred here.

Preferred compositions according to the invention contain purine and/or purine derivatives in relatively narrow ranges of quantities. Preferred cosmetic agents according to the invention here are characterized in that—based on their weight—they contain 0.001 to 2.5 wt. %, preferably 0.0025 to 1 wt. %, particularly preferably 0.005 to 0.5 wt. % and in particular 0.01 to 0.1 wt. % purine(s) and/or purine derivative(s). Preferred cosmetic agents according to the invention are characterized in that they contain purine, adenine, guanine, uric acid, hypoxanthine, 6-purine thiol, 6-thioguanine, xanthine, caffeine, theobromine or theophylline. In hair cosmetic preparations, caffeine is most preferred.

In another preferred embodiment of the present invention, the cosmetic agent contains ectoine ((S)-2-methyl-1,4,5,6-tetrahydro-4-pyrimidinecarboxylic acid.

Particularly preferred according to the invention are agents which—based on their weight—contain 0.00001 to 10.0 wt. %, preferably 0.0001 to 5.0 wt. % and in particular 0.001 to 3 wt. % of the active substances from the group made up of carnitine, taurine, coenzyme Q-10, ectoine, a purine and derivatives thereof, particularly caffeine, or physiologically acceptable salts thereof.

In another embodiment, the agents according to the invention should additionally contain at least one UV light protective filter. UVB filters can be oil-soluble or water-soluble.

The following may be mentioned as examples of oil-soluble substances:
3-benzylidene camphor, e.g. 3-(4-methylbenzylidene)camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as e.g. 2,4,6-trianilino-(p-carbo-2′-ethyl-1′-hexyloxy)-1,3,5-triazine and octyl triazone;
   propane-1,3-diones, such as e.g. 1-(4-tert.butylphenyl)-3-(4′methoxyphenyl)propane-1,3-dione.

The following are suitable as water-soluble substances:
2-phenylbenzimidazole-5-sulfonic acid, and alkali, alkaline earth, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as e.g. 4-(2-oxo-3-bornylidene methyl)benzenesulfonic acid and 2-methyl-5-(2-oxo-3-bornylidene)sulfonic acid and salts thereof.

Typical UV-A filters include, in particular, derivatives of benzoyl methane, such as for example 1-(4′-tert.-butylphenyl)-3-(4′-methoxyphenyl)propane-1,3-dione or 1-phenyl-3-(4′-isopropylphenyl)propane-1,3-dione. The UV-A and UV-B filters can, of course, also be used in mixtures. In addition to the above-mentioned soluble substances, insoluble pigments, in particular finely disperse metal oxides and salts, such as for example titanium dioxide, zinc oxide, iron oxide, aluminum oxide, cerium oxide, zirconium oxide, silicates (talc), barium sulfate and zinc stearate, are also suitable for this purpose. The particles in this case should have an average diameter of less than 100 nm, preferably between 5 and 50 nm and in particular between 15 and 30 nm. They can have a spherical shape, but it is also possible to use those particles that possess an ellipsoid shape or one that deviates from the spherical form in another way.

Finally, further advantages are obtained through the use of plant extracts (L) in the compositions according to the invention. According to the invention, in particular extracts of green tea, oak bark, stinging nettle, witch hazel, hops, henna, chamomile, burdock root, horsetail, whitethorn, lime-tree blossom, almond, aloe vera, pine needles, horse chestnut, sandalwood, juniper, coconut, mango, apricot, lime, wheat, kiwi, melon, orange, grapefruit, sage, rosemary, birch, mallow, valerian, cuckoo flower, wild thyme, yarrow, thyme, melissa, rest harrow, coltsfoot, marshmallow, meristem, ginseng, coffee, cocoa, moringa, ginger root and ayurvedic plant extracts such as, for example, Aegle marmelos (Bilva), Cyperus rotundus (Nagarmotha), Emblica officinalis (Amalaki), Morida citrifolia (Ashyuka), Tinospora cordifolia (Guduchi), Santalum album, (Chandana), Crocus sativus (Kumkuma), Cinnamonum zeylanicum and Nelumbo nucifera (Kamala), sweet grasses, such as wheat, barley, rye, oats, spelt, maize, the various varieties of millet (common millet, finger millet and foxtail as examples), sugar cane, perennial ryegrass, meadow foxtail, tall oatgrass, bentgrass, meadow fescue, purple moor grass, bamboo, cotton grass, pennisetums, Andropogonodeae (Imperata cylindrica, also known as blady grass or cogon grass), buffalo grass, cord-grasses, dog's tooth grasses, lovegrasses, Cymbopogon (lemon grass), Oryzeae (rice), Zizania (wild rice), beach grass, blue oat grass, soft grasses, quaking grasses, meadow grasses, couch grass and echinacea, in particular Echinacea angustifolia DC, Echinacea paradoxa (Norton), Echinacea simulata, E. atrorubens, E. tennesiensis, Echinacea strigosa (McGregor), Echinacea laevigata, Echinacea purpurea (L.) Moench and Echinacea pallida (Nutt), all types of vines and pericarp of Litchi chinensis are preferred.

The plant extracts can be used according to the invention both in pure form and in dilute form. Where they are used in dilute form, they usually contain approx. 2-80 wt. % active substance and, as solvent, the extracting agent or mixture of extracting agents used to obtain them.

Furthermore, the cosmetic agents can contain additional active substances, auxiliary substances and additives, such as for example
structurants, such as maleic acid and lactic acid,
swelling agents, such as urea, allantoin, carbonates or hydantoin,
dimethyl isosorbide and cyclodextrins,
dyes for coloring the agent,
active anti-dandruff substances, such as piroctone olamine, zinc omadine and climbazole,
complexing agents, such as EDTA, NTA, β-alanine diacetic acid and phosphonic acids,
opacifiers, such as latex, styrene/PVP and styrene/acrylamide copolymers,
pearlescent agents, such as ethylene glycol mono- and distearate and PEG-3 distearate,
pigments,
stabilizers for hydrogen peroxide and other oxidizing agents,
propellants, such as propane-butane mixtures, N₂O, dimethyl ether, CO₂ and air,
antioxidants,
perfume oils, fragrances and odorants.

With regard to further optional components and the amounts of these components used, express reference is made to the relevant handbooks known to the person skilled in the art.

As already mentioned, particular importance is attached to the high conditioning action of the agents according to the invention. Preferred agents according to the invention also contain in a cosmetic support, in addition to a cationic amino-functional silicone, most preferably Silicone Quaternium-22, and a B-series vitamin, one of the following combinations of active substances (i) to (xxxv):
(i) at least taurine or at least one derivative of taurine,
(ii) at least one purine, in particular caffeine,
(iii) at least carnitine,
(iv) at least ectoine,
(v) at least one ubiquinone, in particular coenzyme Q-10,
(vi) at least one cationic surfactant,
(vii) at least one cationic polymer,
(viii) at least one additional silicone,
(ix) at least one UV-absorbing agent,
(x) at least one plant extract, in particular selected from the extracts of hops, ginseng, Litchi chinensis or echinacea,
(xi) ectoine and at least one cationic surfactant,
(xii) ectoine and at least one cationic polymer,
(xiii) ectoine and at least one silicone,
(xiv) ectoine and at least one UV-absorbing agent,
(xv) ectoine and at least one additional plant extract, in particular selected from the extracts of hops, ginseng, Litchi chinensis or echinacea,
(xvi) carnitine and at least one cationic surfactant,
(xvii) carnitine and at least one cationic polymer,
(xviii) carnitine and at least one silicone,
(xix) carnitine and at least one UV-absorbing agent,
(xx) carnitine and at least one additional plant extract, in particular selected from the extracts of hops, ginseng, Litchi chinensis or echinacea,
(xxi) coenzyme Q-10 and at least one cationic surfactant,
(xxii) coenzyme Q-10 and at least one cationic polymer,
(xxiii) coenzyme Q-10 and at least one silicone,
(xxiv) coenzyme Q-10 and at least one UV-absorbing agent,
(xxv) coenzyme Q-10 and at least one additional plant extract, in particular selected from the extracts of hops, ginseng, Litchi chinensis or echinacea,
(xxvi) caffeine and at least one cationic surfactant,
(xxvii) caffeine and at least one cationic polymer,
(xxviii) caffeine and at least one silicone,
(xxix) caffeine and at least one UV-absorbing agent,
(xxx) caffeine and at least one additional plant extract, in particular selected from the extracts of hops, ginseng, Litchi chinensis or echinacea,
(xxxi) taurine and at least one cationic surfactant,
(xxxii) taurine and at least one cationic polymer,
(xxxiii) taurine and at least one silicone,
(xxxiv) taurine and at least one UV-absorbing agent,
(xxxv) taurine and at least one additional plant extract, in particular selected from the extracts of hops, ginseng, Litchi chinensis or echinacea.

The present invention also provides a method for hair treatment in which a cosmetic agent according to claim 1 is applied on to the hair and rinsed off the hair after a period of exposure.

The period of exposure is preferably a few seconds to 100 minutes, particularly preferably 1 to 50 minutes and most particularly preferably 1 to 30 minutes.

Furthermore, a method in which a cosmetic agent according to claim 1 is applied on to the hair and remains there is also according to the invention. The term “to remain on the hair” is understood according to the invention to mean that the agent is not rinsed out of the hair again immediately after its application. In this case, the agent instead remains on the hair for more than 100 minutes up to the next hair wash.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for the treatment of keratinic fibers, in particular human hair, comprising:
a. at least one cationic amino-functional silicone;
b. at least one vitamin from the B series; and
c. a cosmetic support.

2. The cosmetic composition according to claim 1, wherein the cationic amino-functional silicone is Silicone Quaternium-22.

3. The cosmetic composition according to claim 1, wherein the vitamin from the B series is selected from the group consisting of panthenol, pantolactone, pantothenic acid, pyridoxine and derivatives thereof, and nicotinamide.

4. The cosmetic composition according to claim 1, wherein cationic amino-functional silicone is included in an amount of 0.01 to 20.0 wt. %.

5. The cosmetic composition according to claim 1, further comprising at least one additional ingredient selected from the group consisting of a cationic surfactant, a cationic polymer, and an amphoteric polymer.

6. The cosmetic composition according to claim 1, further comprising at least one further silicone selected from the group consisting of dimethicones, amodimethicones and dimethiconols.

7. The cosmetic composition according to claim 6, wherein the further silicone has a viscosity of 40,000 to 80,000 mPa·s.

8. The cosmetic composition according to claim 1, further comprising at least one active substance selected from the group consisting of carnitine, taurine, coenzyme Q-10, ectoine, and a purine or a derivative or physiologically acceptable salt thereof.

9. A method for the treatment of keratinic fibers, comprising:
applying a cosmetic composition according to claim 1 on to the keratinic fibers; and
rinsing the hair after an exposure period ranging between a few seconds and 45 minutes.
